(19) United States
(12) Plant Patent Application Publication
(10) Pub. No.: US 2004/0088770 P1
(43) Pub. Date: May 6, 2004

(54) Fothergilla major plant named 'Blue Shadow'

(76) Inventor: Gary Handy, Sandy, OR (US)

Correspondence Address: KLARQUIST SPARKMAN, LLP, 121 SW SALMON STREET, SUITE 1600, PORTLAND, OR, 97204, US

(21) Appl. No.: 10/288,680

(22) Filed: Nov. 4, 2002

(51) Int. Cl. A01H 5/00

(52) U.S. Cl. PLT/226

(57) ABSTRACT

A new and distinct cultivar of Fothergilla major plant characterized by its striking glaucous, blue green leaf color.

### FOTHERGILLA MAJOR PLANT NAMED ‘BLUE SHADOW’

Botanical classification: Fothergilla major

Cultivar name: ‘Blue Shadow’

The present invention relates to a new and distinct cultivar of deciduous shrub of the genus and species, Fothergilla major, and given the cultivar name ‘Blue Shadow’. The genus Fothergilla is of the family Hamamelidaceae.

The new cultivar was found in a cultivated area by Gary Handy as a basal shoot mutation (bud sport) from a young plant of Fothergilla major ‘Mt. Airy’, an unpatented plant.

Fothergilla major, is one of two species of Fothergilla native to southeastern USA. These deciduous shrubs are grown for their spring flowers and outstanding autumn foliage color. Fothergilla major, or Mountain Witch Hazel, as described in Botanica, The Illustrated A to Z of over 10,000 Garden Plants, is “the best known of the genus and grows to 1.8 to 3 m tall and nearly as wide. Fragrant, white, puffball flowers appear in spring and again in autumn. The dark green leaves, slightly blue beneath, turn vibrant yellows, oranges and reds in the fall.”

This new cultivar is characterized by the following:

1. Unique, glaucous blue green leaves.

2. Rounder leaves than parent.

3. Good fall color.

4. Excellent vigor.

Asexual propagation by cuttings in Boring, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

FIG. 1 shows a close up of the new cultivar.

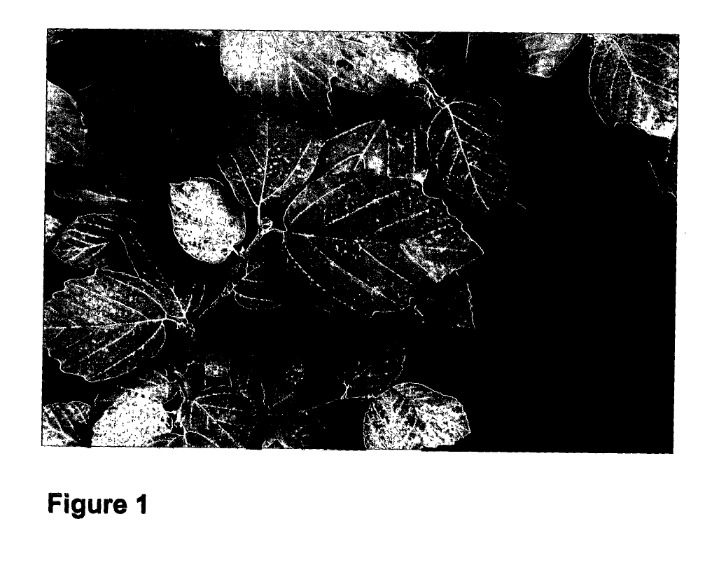

FIGS. 2 and 3 show the overall shape of the plant.

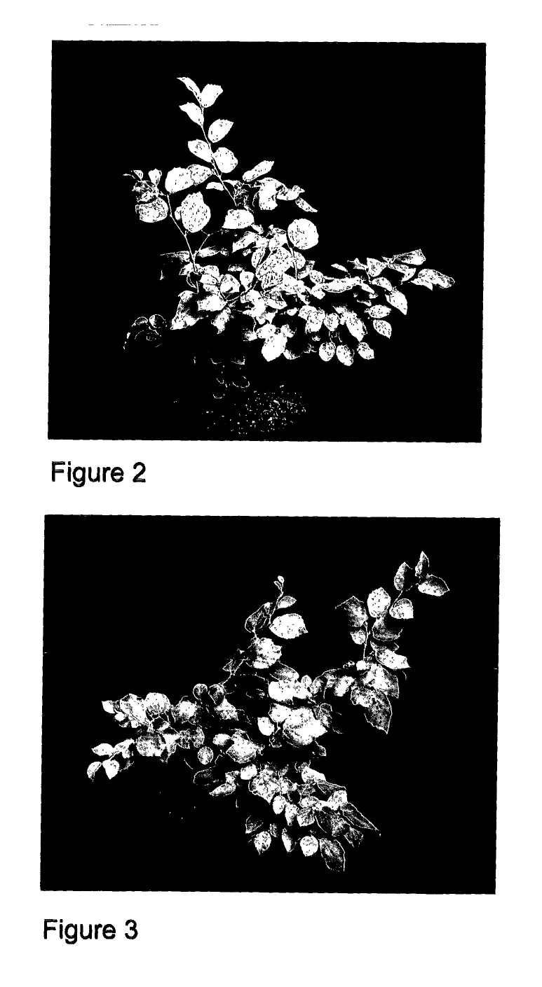

### DETAILED PLANT DESCRIPTION

The following is a detailed description of the new cultivar based on observations of a three-year-old specimen grown in 3-gallon pots grown in shade with high nutrition in Boring, Oreg. The color descriptions are all based on The Royal Horticultural Society Colour Chart.

Plant:

Type.—Deciduous shrub.

Hardiness.—USDA Zones 4 to 9.

Size.—1.5 to 3 m high and wide.

Form.—Globose.

Stem:

Type.—Numerous, usually erect, same as Fothergilla major ‘Mt. Airy’.

Size.—Three-year stem is 1.3 cm wide.

Surface.—Young branches are glaucous and have stellate pubescence.

Color from dormant plant.—1 year old stem is Greyed Green 199C. 2-year-old stem is Greyed Green 197C and Greyed Green Group 188A.

Dormant buds:

Size.—Flower buds are 0.65 cm long and 0.5 cm wide. Leaf buds are 0.4 cm long and 0.2 cm wide.

Surface.—Thick with stellate pubescence.

Color.—Closest to 199C but somewhat pinker.

Leaf:

Type.—Simple.

Shape.—Broadly elliptic.

Arrangement.—Alternate.

Length.—4.5 to 7 cm.

Width.—3.5 to 5.5 cm. at the widest part.

Vestiture.—Stellate pubescent and glaucous.

Aspect.—Matte.

Texture.—Leathery.

Venation.—Pinnate, main vein is sometimes pulled off center near the leaf tip due to leaf puckering.

Margins.—Entire on lower ⅔, irregularly and coarsely serrate on top ⅓.

Apex.—Obtuse.

Base.—Oblique.

Petiole length.—0.7 cm.

Petiole texture.—Glaucous and stellate pubescent.

Petiole color.—Yellow Green 147B to Greyed Brown 199D.

Color.—Topside — Greyed Green Group 188A and Black Group 202C. New Growth — Greyed Green 189B. Bottom — Green Group 138A.

Flower, fruit and seed.—Not yet observed. This plant has not flowered yet.

### Comparisons to Similar Fothergilla

Compared to Fothergilla major ‘Mt. Airy’ (an unpatented plant), this new cultivar has a completely different leaf color and shape. Its leaves are a blue green rather than green. Its leaf shape is rounder. Because this new cultivar is so glaucous its surface is not glossy like Fothergilla major ‘Mt. Airy’. This glaucous covering extends to the young branches making them a different color as well. Their habits, growth rate, and eventual size are the same. Both have excellent fall color.

We claim:

1. A new and distinct cultivar of Fothergilla major plant as shown and described, characterized by its striking glaucous, blue green leaf color.
